(19) United States
(12) Patent Application Publication
(10) Pub. No.: US 2026/0274782 A1
(43) Pub. Date: Sep. 17, 2026

(54) METHOD FOR SEPARATING DEPOLYMERIZATION PRODUCT

(71) Applicant: HONDA MOTOR CO., LTD., Tokyo (JP)

(72) Inventor: Ryo NAKAI, Saitama (JP)

(21) Appl. No.: 19/560,155

(22) Filed: Mar. 9, 2026

(30) Foreign Application Priority Data
Mar. 12, 2025 (JP) 2025-039564

(51) Int. Cl. C07C 51/48

(52) U.S. Cl. CPC C07C 51/48

(57) ABSTRACT

Provided is a technology that enables separating and recycling a depolymerization product of a blend resin including polyester resin having a terephthalic acid unit. A method for separating a depolymerization product includes a neutralization step including neutralizing an aqueous solution with a strong acid that is stronger than terephthalic acid, the aqueous solution including a depolymerization product after a depolymerization reaction of a blend resin including polyester resin having a terephthalic acid unit; and a liquid separation step including adding an ester-based extraction solvent to the aqueous solution after the neutralization, thereby performing liquid separation. The strong acid is preferably sulfuric acid or hydrochloric acid.

This application is based on and claims the benefit of priority from Japanese Patent Application No. 2025-039564, filed on 12 Mar. 2025, the content of which is incorporated herein by reference.

### BACKGROUND OF THE INVENTION

### Field of the Invention

The present invention relates to a method for separating a depolymerization product.

### Related Art

In recent years, efforts to significantly reduce waste intensified through recycling waste. To this end, research and development of recycling of recovered resin is being carried out.

For example, Japanese Unexamined Patent Application, Publication No. 2023-163661 discloses a method for recovering terephthalic acid. The method includes a depolymerization reaction step including depolymerizing a crushed product of polyethylene terephthalate resin in an alkaline solution, and a neutralization crystallization step including adding an acid having an acid dissociation constant of 2.9 or more as a neutralizing acid to a treatment liquid obtained by the depolymerization reaction and neutralizing and crystallizing the treatment liquid to precipitate terephthalic acid.
Patent Document 1: Japanese Unexamined Patent Application, Publication No. 2023-163661

### SUMMARY OF THE INVENTION

Incidentally, since recovered resin is often a blend resin in which a plurality of kinds of resins are mixed, a technology capable of separating and recycling the depolymerization product of the blend resin is required.

The present invention has an object to provide a technology that enables separating and recycling a depolymerization product of a blend resin including polyester resin having a terephthalic acid unit.
(1) A method for separating a depolymerization product includes: a neutralization step including neutralizing an aqueous solution with a strong acid that is stronger than terephthalic acid, the aqueous solution including a depolymerization product after a depolymerization reaction of a blend resin including polyester resin having a terephthalic acid unit; and a liquid separation step including adding an ester-based extraction solvent to the aqueous solution after the neutralization, thereby performing liquid separation.
(2) In the method for separating a depolymerization product described in (1), the strong acid is sulfuric acid or hydrochloric acid.
(3) The method for separating a depolymerization product described in (1) or (2) further includes an acid precipitation step including adding a strong acid to an aqueous phase after the liquid separation to precipitate acid, and the strong acid used in the neutralization step is identical to the strong acid used in the acid precipitation step.
(4) In the method for separating a depolymerization product described in any one of (1) to (3), in the neutralization step, pH of the aqueous solution including the depolymerization product is adjusted to 6.5 or more and 7.5 or less.
(5) The method for separating a depolymerization product described in any one of (1) to (4) further includes, before the neutralization step, a water stirring step including adding water to the depolymerization product obtained by removing a solvent from a reaction liquid after the depolymerization reaction and performing stirring.

The present invention can provide a technology that enables separating and recycling a depolymerization product of a blend resin including polyester resin having a terephthalic acid unit.

### BRIEF DESCRIPTION OF THE DRAWINGS

FIG. 1 is a diagram schematically showing a configuration of a blend resin regeneration apparatus according to one embodiment of the present invention; and

FIG. 2 is a flowchart showing a process procedure of a method for regenerating the blend resin according to one embodiment of the present invention.

### DETAILED DESCRIPTION OF THE INVENTION

Hereinafter, one embodiment of the present invention is described in more detail with reference to the drawings.

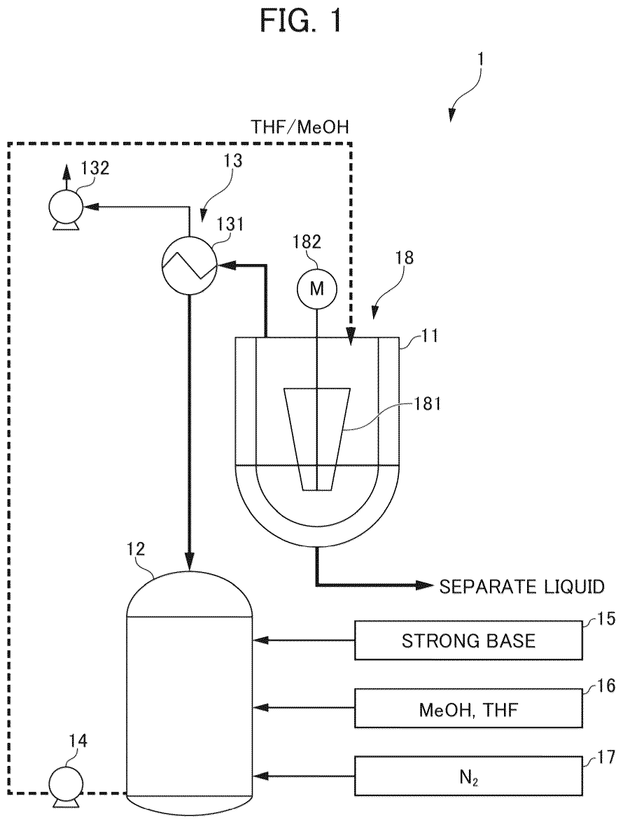

A blend resin regeneration apparatus according to the present embodiment is an apparatus capable of depolymerizing a blend resin including polyester resin having a terephthalic acid unit, and separating and recycling a depolymerization product after the depolymerization. FIG. 1 is a diagram schematically showing a configuration of a blend resin regeneration apparatus 1 according to the present embodiment. As shown in FIG. 1, the blend resin regeneration apparatus 1 according to the present embodiment includes a depolymerization tank 11, a storage tank 12, a recovery device 13, a liquid feeder 14, a first supplier 15, a second supplier 16, a purge device 17, and a stirring device 18. Note here that an open/close valve or the like (not shown) is provided in a piping path linking these component devices, and the regeneration apparatus 1 is driven and controlled by a control device (not shown) for controlling the component devices and the open/close valve or the like, and the blend resin is regenerated.

The blend resin used in the present embodiment include polyester resin having a terephthalic acid unit, and may be a mixture of the polyester resin having a terephthalic acid unit and a plurality of kinds of resins other than the polyester resin having a terephthalic acid unit. The blend resin may include at least one of a polyurethane (PU) resin, a polyethylene (PE) resin, a polypropylene (PP) resin, a polyamide (PA) resin such as nylon, cotton, an acrylic resin, wool and rayon, in addition to the polyester resin. Examples of the polyester resin having a terephthalic acid unit include polyethylene terephthalate (PET). Furthermore, the blend resin may be a blend fiber composed of resin fibers. As the blend resin, for example, a resin derived from an end of life vehicle (ELV) or a resin derived from clothing is used.

The depolymerization tank 11 allows a blend resin including polyester resin having a terephthalic acid unit to be depolymerized in, for example, a solution in which a strong base is dissolved in a solvent including THE and methanol. The depolymerization temperature is not particularly limited, and is, for example, 45° C. or more and 75° C. or less. Furthermore, the depolymerization time is not particularly limited, and, for example, 5 hours or more and 20 hours or less.

Methanol used as a solvent for depolymerization in the present embodiment is considered to have a function as a reaction solvent for transesterification of polyester resin, and a function as a solvent for dissolving a strong base. Specifically, it is considered that CH₃O⁻ generated by the strong base reacts with the polyester resin, and OH⁻ and CH₃O⁻ compete with each other. For example, when water and a strong base are used, the affinity (solubility) with the polyester resin is poor, so that a high temperature is required to allow the reaction to proceed; and THE and water are not compatible, so that the reaction is considered to be difficult to occur.

The depolymerization tank 11 is provided with the stirring device 18 for stirring a reaction liquid in the depolymerization tank 11. The stirring device 18 includes a stirrer 181 for stirring the reaction liquid in the depolymerization tank 11, and a motor 182 for rotationally driving the stirrer 181. The reaction liquid in the depolymerization tank 11 is stirred by the stirring device 18, raw materials are well mixed, and the depolymerization reaction proceeds efficiently.

The storage tank 12 stores a solvent recovered by a recovery device 13 described later from the reaction liquid after depolymerization of the blend resin. Examples of the recovered solvents include THF and methanol.

The recovery device 13 recovers a solvent from the reaction liquid inside the depolymerization tank 11 after the blend resin is depolymerized. Specifically, the recovery device 13 includes a condenser 131 and a vacuum pump 132. The vacuum pump 132 depressurizes the inside of the depolymerization tank 11 after the depolymerization, promotes vaporization of the solvent in the reaction liquid at a high temperature (for example, 40° C. or more and 75° C. or less) after depolymerization, and introduces the solvent into the condenser 131. The condenser 131 cools and condenses the introduced gas solvent to be liquefied, and the liquid is then fed to the storage tank 12.

Note here that the solvent recovered by the recovery device 13 includes, for example, THF and methanol, but the ratio thereof is different from that in a charging raw material. Specifically, in the depolymerization of polyester resin having a terephthalic acid unit, since methanol is used also for the methyl esterification of a carboxylic acid, the content ratio of methanol in the recovered solvent is smaller than that in the charging raw material.

The liquid feeder 14 feeds a solution, which is stored in the storage tank 12 and in which a strong base is dissolved in a solvent, to the depolymerization tank 11. Specifically, the liquid feeder 14 includes a liquid feeding pump. The solvent for depolymerization is recycled by the liquid feeder 14.

The first supplier 15 supplies a strong base into the storage tank 12. The strong base is not particularly limited as long as the strong base can depolymerize polyester resin having a terephthalic acid unit, and examples include sodium hydroxide, potassium hydroxide, barium hydroxide, strontium hydroxide, and lithium hydroxide. Among them, sodium hydroxide is preferably used. The first supplier 15 supplies the strong base so that the concentration of the strong base dissolved in the solvent in the storage tank 12 is, for example, 0.02 g/mL or more and 0.14 g/mL or less.

The second supplier 16 supplies methanol and THE to the solvent recovered by the above-described recovery device 13. As described above, the solvent recovered by the recovery device 13 includes, for example, THF and methanol, but the ratio of them is different from that in the charging raw material. Specifically, the content ratio of the methanol in the recovered solvent is smaller than that in the charging raw material. Therefore, the second supplier 16 supplies and compensates a shortage of methanol so that the methanol ratio in the recovered and stored solvent coincide with that in the charging raw material. Thus, since methanol is compensated before the solvent for depolymerization is recovered and reused, the charging amount of the raw material can be made constant. Furthermore, since the recovery of THE by the recovery device 13 is not complete, THE is also compensated by the second supplier 16.

FIG. 1 shows an example in which the second supplier 16 supplies methanol and THE into the storage tank 12. In this case, the second supplier 16 can share a supply unit such as supply pump (not shown) with the above-described first supplier 15.

The purge device 17 purges the inside of the storage tank 12 with an inert gas. In the example shown in FIG. 1, the purge device 17 supplies nitrogen N₂ as the inert gas to the inside of the storage tank 12, and the air in the storage tank 12 is purged with nitrogen N₂. This can suppress contamination of air into a solvent that can be generated by the supply of a strong base by the first supplier 15, and by the supply of methanol by the second supplier 16.

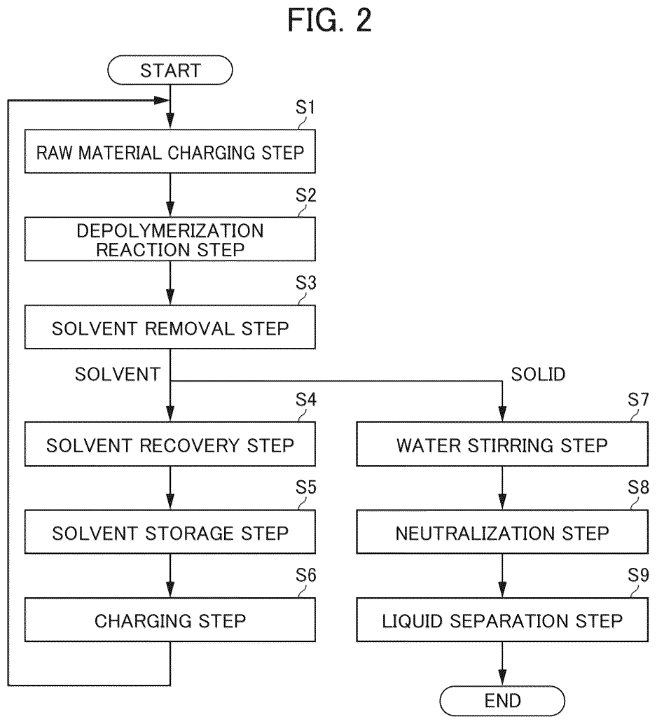

Next, a method for regenerating a blend resin according to the present embodiment will be described. FIG. 2 is a flowchart showing a process procedure of the method for regenerating the blend resin according to the present embodiment. In FIG. 2, processes of Steps S1 to S6 are executed by the above-described regeneration apparatus 1. Furthermore, processes of Steps S7 to S9 are related to a method for separating a depolymerization product. In other words, the method for separating a depolymerization product according to the present embodiment includes a water stirring step, a neutralization step, and liquid separation step.

In a raw material charging step as Step S1, a process of charging raw material is executed. Specifically, in addition to the blend resin including polyester resin having a terephthalic acid unit, for example, a mixed solvent of THE and methanol for depolymerization and sodium hydroxide as a strong base are loaded into the depolymerization tank 11 at a predetermined ratio, and mixed by stirring with the stirring device 13 to obtain a raw material.

In a depolymerization reaction step as Step S2, a depolymerization reaction process of the blend resin is executed in the depolymerization tank 11. For example, the depolymerization reaction is allowed to proceed at a depolymerization temperature of 45° C. or more and 75° C. or less, and a depolymerization time of 5 hours or more and 20 hours or less.

In a solvent removal step as Step S3, after the depolymerization reaction, a process of removing the solvent from the reaction liquid at a high temperature (for example, 40° C. or more and 75° C. or less) is executed in the depolymerization tank 11. Specifically, the pressure in the depolymerization tank 11 is reduced by the vacuum pump 132 of the recovery device 13, and vaporization of the solvent is promoted to remove the solvent.

In a solvent recovery step as Step S4, a process of recovering the solvent removed in Step S3 is executed. Specifically, the solvent vaporized and removed is recovered by condensing and liquefying the solvent by the condenser 131 of the recovery device 13.

In a solvent storage step as Step S5, the solvent recovered in Step S4 is introduced into and stored in the storage tank 12. The recovered solvent is stored in the storage tank 12 for reuse in the next depolymerization reaction of the blend resin.

In a charging step as Step S6, a strong base, for example, sodium hydroxide is supplied to the recovered and stored solvent. Furthermore, methanol is supplied to the recovered and stored solvent for compensation, and the methanol ratio is adjusted so as to coincide with that in the charging raw material. At this time, a purging process of purging the inside of the storage tank 12 with an inert gas (nitrogen N₂) is also executed by the purge device 17.

After the charging step, the solvent stored in the storage tank 12 is subjected to the raw material charging step of Step S1 to be reused in the next depolymerization of the blend resin. Thus, the solvent for depolymerization is reused.

Next, processes of Steps S7 to S9, which are related to the method for separating a depolymerization product according to the present embodiment, will be described. Note here that a water stirring step as Step S7 is not an essential step. Separation methods not including the water stirring step are also included in the method for separating a depolymerization product according to the present embodiment.

The water stirring step as Step S7 executes a water stirring process, in which water is added to a depolymerization product, that is, a solid from which the solvent has been removed in the solvent removal step of Step S3, and stirring is performed. Specifically, an appropriate amount of ion-exchanged water is added to the depolymerization product as the solid, and stirring process is executed, for example, at room temperature for 1 hour.

As described above, the method for separating a depolymerization product according to the present embodiment may further include, before a neutralization step described later, the water stirring step in which water is added to the depolymerization product obtained by removing the solvent from the reaction liquid after the depolymerization reaction, followed by stirring. When the water stirring step is included, the reaction represented by the following reaction formula (1) can be promoted, and monomethyl terephthalate, which is a depolymerization product of polyester resin having a terephthalic acid unit, can be changed into terephthalic acid.

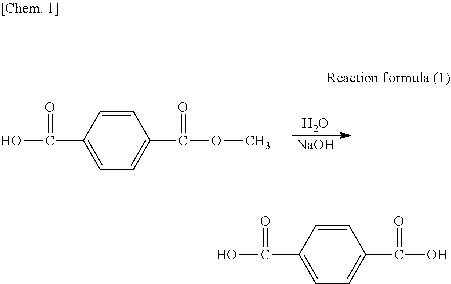

In the neutralization step as Step S8, a neutralization process is executed to the aqueous solution obtained in Step 7. Specifically, a strong acid, for example, an appropriate amount of 2N sulfuric acid aqueous solution is added to the aqueous solution obtained in Step 7, and the solution is stirred and neutralized so that the pH thereof becomes around 7. Note here that when this neutralization step is included, it is possible to suppress a problem that an ester-based extraction solvent is decomposed and impurities are formed in a liquid separation step as a subsequent process. This will be described in detail later.

In the liquid separation step as Step S9, a liquid separation process is executed to the aqueous solution neutralized in Step S8. Specifically, an appropriate amount of an ester-based extraction solvent is added to the aqueous solution neutralized in Step S8, followed by stirring and liquid separation.

Herein, examples of the ester-based extraction solvent include ethyl acetate, methyl acetate, propyl acetate, isopropyl acetate, butyl acetate, isobutyl acetate, isopropyl acetate, sec-butyl acetate, ethyl 3-ethoxypropionate, diethylene glycol monoethyl ether acetate, 2-ethylhexyl acetate, isoamyl acetate, and the like. Among them, ethyl acetate is preferably used.

By repeating the liquid separation operation a plurality of times, the liquid is separated into an aqueous phase and an oil phase. Terephthalic acid as a depolymerized product of polyester resin having a terephthalic acid unit can be obtained from the aqueous phase, and a depolymerized product of a polyurethane resin or the like can be obtained from the oil phase. As mentioned above, the process of the blend resin regeneration method is completed.

Next, the reason why when this neutralization step is included, it is possible to suppress a problem that the ester-based extraction solvent is decomposed and impurities are formed in the liquid separation step as a subsequent process will be described in detail below.

Firstly, when the aqueous solution including the depolymerization product is alkaline, a problem occurs in which the ester-based extraction solvent is hydrolyzed and impurities are formed in the liquid separation step as the subsequent process. For example, when ethyl acetate is used as the ester-based extraction solvent, when the aqueous solution including the depolymerization product is alkaline, the following reaction formula (2) proceeds, and ethyl acetate is hydrolyzed into sodium acetate and methanol, and impurities are formed.

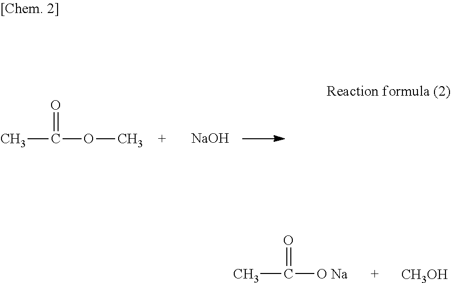

Thus, the present embodiment includes, before the liquid separation step, the neutralization step of neutralizing an aqueous solution including the depolymerization product after the depolymerization reaction with an acid stronger than terephthalic acid. Thus, since the aqueous solution including the depolymerization product after the depolymerization reaction is neutralized and becomes substantially neutral, the progress of the reaction represented by the reaction formula (2) can be suppressed, and the problem that the ester-based extraction solvent is decomposed and impurities are formed can be suppressed.

The strong acid used in the neutralization step may be any strong acid as long as the acid is stronger than terephthalic acid. Any strong acid with a lower acid dissociation constant than the acid dissociation constant pKa₁ of terephthalic acid of 2.75 can be used. Examples thereof include sulfuric acid, hydrochloric acid, nitric acid, phosphoric acid, methanesulfonic acid, benzenesulfonic acid, toluenesulfonic acid, and the like. Among them, sulfuric acid or hydrochloric acid is preferably used. Using sulfuric acid or hydrochloric acid as the strong acid is preferable because countermeasures against reaction products such as reaction gas accompanying neutralization are simple.

In the neutralization step, the pH of the aqueous solution including the depolymerization product is preferably adjusted to 6.5 or more and 7.5 or less by using the strong acid. When the pH of the aqueous solution including the depolymerization product is 6.5 or more, acid precipitation of the depolymerization product can be suppressed. When the pH of the aqueous solution including the depolymerization product is 7.5 or less, hydrolysis and impurity formation of the ester-based extraction solvent can be suppressed.

Note here that an acid precipitation step (not shown) of adding a strong acid to the aqueous phase after the liquid separation step may further be included, and the strong acid used in the neutralization step is preferably the same as the strong acid used in the acid precipitation step. This can reduce the types of impurities, and can facilitate purification of repellents substances at the time of depolymerization.

The embodiment of the present invention has been described above, but the present invention is not limited to the embodiment described above, and the embodiment may be appropriately modified within the scope of the spirit of the present invention.

### Examples

Hereinafter, Example of the present invention is described, but the present invention is not limited to this Example.

Firstly, in addition to PET as polyester resin having a terephthalic acid unit, blend resin including PU resin, PP resin, PE resin, nylon 6 (PA6) as a PA resin, and cotton was prepared. To 4 g of the blend resin, 50 mL of new tetrahydrofuran, 50 mL of methanol, and 2 g of sodium hydroxide were added and the mixture was loaded into the depolymerization tank 11. After the loading, the mixture was stirred at 65° C. for 20 hours to depolymerize the blend resin.

After the depolymerization, the solvent was recovered by a recovery device and stored, and sodium hydroxide was supplied. Furthermore, methanol was supplied so that the ratio of methanol was equal to that of the solvent of the charging raw material. Then, the solvent was subjected to the next depolymerization reaction.

On the other hand, 150 mL of water was added to a depolymerization product as a solid from which the solvent had been distilled off, and the mixture was stirred at room temperature for 1 hour. After stirring, 2N sulfuric acid aqueous solution was added until pH became 6.5 to 7.5, then 150 mL of ethyl acetate was added, and the mixture was stirred at room temperature for 5 minutes.

Then, an insoluble material was filtered under reduced pressure with a Buchner funnel, a filtrate was washed with a mixed solvent (20 ml of water/20 ml of ethyl acetate), and dried at 80° C. overnight to recover a mixture of PP resin, PE resin, PA6, and cotton.

Then, the filtrate was introduced into a liquid separation funnel, and an operation of extracting the PU depolymerized product into 150 mL of ethyl acetate was repeated three times to recover the aqueous phase and the oil phase, respectively.

After 150 mL of saturated saline was added to the oil phase, the oil phase was washed and recovered using a liquid separation funnel. Thereafter, magnesium sulfate was added so as to dry the oil phase, followed by filtration under reduced pressure, and the magnesium sulfate was washed with 20 mL of ethyl acetate.

Then, the solvent was distilled off from the filtrate by using an evaporator and dried at 80° C. overnight to recover the PU depolymerized product (polyol PO).

On the other hand, 2N sulfuric acid aqueous solution was added to the aqueous phase until the pH became 2 to precipitate terephthalic acid (TPA), followed by filtration under reduced pressure. Finally, the filtrate was washed with 20 mL of methanol and dried at 80° C. overnight to recover TPA.

As mentioned above, according to this Example, it has been demonstrated that a blend resin including polyester resin having a terephthalic acid unit can be depolymerized, and the depolymerization product after depolymerization can be separated and recycled. Thus, the effect of the present invention was verified.

### EXPLANATION OF REFERENCE NUMERALS

1 regeneration apparatus
11 depolymerization tank
12 storage tank
13 recovery device
131 condenser
132 vacuum pump
14 liquid feeding pump (liquid feeder)
15 first supplier
16 second supplier
17 purge device
19 stirring device
191 stirrer
192 motor

What is claimed is:

1. A method for separating a depolymerization product, the method comprising:
a neutralization step including neutralizing an aqueous solution with a strong acid that is stronger than terephthalic acid, the aqueous solution comprising a depolymerization product after a depolymerization reaction of a blend resin comprising polyester resin having a terephthalic acid unit; and
a liquid separation step including adding an ester-based extraction solvent to the aqueous solution after the neutralization, thereby performing liquid separation.

2. The method for separating a depolymerization product according to claim 1, wherein the strong acid is sulfuric acid or hydrochloric acid.

3. The method for separating a depolymerization product according to claim 1, further comprising
an acid precipitation step including adding a strong acid to an aqueous phase after the liquid separation to precipitate acid; wherein
the strong acid used in the neutralization step is identical to the strong acid used in the acid precipitation step.

4. The method for separating a depolymerization product according to claim 1, wherein in the neutralization step, pH of the aqueous solution comprising the depolymerization product is adjusted to 6.5 or more and 7.5 or less.

5. The method for separating a depolymerization product according to claim 1, further comprising, before the neutralization step, a water stirring step including adding water to the depolymerization product obtained by removing a solvent from a reaction liquid after the depolymerization reaction and performing stirring.
